(19) United States
(12) Patent Application Publication
(10) Pub. No.: US 2019/0345500 A1
(43) Pub. Date: Nov. 14, 2019

(54) METHODS AND PHARMACEUTICAL COMPOSITIONS FOR MODULATING STEM CELLS PROLIFERATION OR DIFFERENTIATION

(71) Applicants: |NSERM (INSTITUT NATIONAL DE LA SANTÉ ET DE LA RECHERCHE MÉDICALE), Paris (FR); UNIVERSITÉ DE NANTES, Nantes (FR); CENTRE NATIONAL DE LA RECHERCHE SCIENTIFIQUE (CNRS), Paris (FR); CENTRE HOSPITALIER UNIVERSITAIRE DE NANTES, Nantes (FR); HCS PHARMA, Loos (FR)

(72) Inventors: Karim SI TAYEB, Nantes - Cedex 1 (FR); Salam IDRISS, Beirut (LB); Bertrand CARIOU, Nantes - Cedex 1 (FR); Méryl ROUDAUT, Nantes - Cedex 1 (FR); Cédric LE MAY, Nantes - Cedex 1 (FR); Amandine CAILLAUD, Nantes - Cedex 1 (FR)

(21) Appl. No.: 16/349,337

(22) PCT Filed: Nov. 14, 2017
(86) PCT No.: PCT/EP2017/079190

§ 371 Date: May 13, 2019

(30) Foreign Application Priority Data
Nov. 14, 2016 (EP) 16306485.0

(51) Int. Cl. C12N 15/113; A61P 35/00

(52) U.S. Cl. CPC C12N 15/1137; A61P 35/00; C12N 2310/531; C12N 2310/14; C12Y 304/21061

(57) ABSTRACT

The present invention relates to a method for modulating stem cells proliferation or differentiation comprising a step of contacting said stem cells with an effective amount of an activator or inhibitor of a proprotein convertase subtilisin kexin 9 (PCSK9). Inventors performed a global transcriptomic analyses in hiPSCs and showed that PCSK9 inhibition by shRNA and the intracellular PCSK9-R104C/V114A mutation negatively regulate the NODAL signaling pathway and its targets. This regulation was manifested in drastic reduction P-SMAD2/total SMAD2 protein level. This was accompanied by reduced proliferation rate where hiPSC-shPCSK9 and hiPSC-R104C/V114A demanded >1.3-fold more time to double compared to their control counterparts. They showed that PCSK9 was regulating this signaling pathway through direct physical interaction with DACT2, an intracellular attenuator of NODAL receptor and favoring its protein degradation. Thus, these findings allow to understand the differentiation and proliferation of cells.

### FIELD OF THE INVENTION

The invention relates to the field of stem cells. More particularly, the invention relates to a method for modulating stem cells proliferation or differentiation by inhibiting or activating PCSK9.

### BACKGROUND OF THE INVENTION

The proprotein convertase subtilisin kexin 9 (PCSK9) was identified and characterized as the ninth member of the proprotein convertase (PC) family (Seidah et al. 2003). That same year has witnessed another blessed milestone when the group of Abifadel identified mutations in the PCSK9 gene in French families with autosomal dominant hypercholesterolemia (ADH) (Abifadel et al. 2003). Since that moment, plethora of studies was conducted to decipher all the aspects of PCSK9 biology.

Unlike other PC members, secreted PCSK9 is enzymatically inactive due to the intact tight association of its pro-domain with the catalytic domain (Cunningham et al. 2007). PCSK9 turned out to be a major regulator of plasma low-density lipoprotein cholesterol (LDL-C) levels through inducing the degradation of hepatic LDL receptors (LDLR) via a non-enzymatic fashion (Seidah 2016). Several gain-of-function (GOF) and loss-of-function (LOF) mutations were identified, which were associated with high LDL-C due to enhanced LDLR degradation and low LDL-C levels with higher hepatic cell surface LDLR respectively (Abifadel et al. 2009; Cohen et al. 2006).

All the knowledge on PCSK9 biology we have in hands so far has been deduced from clinical data, transgenic and knockout animal models, and cell lines. Despite the many advantages of these models, still they are short to fill in the gaps in our insights on PCSK9 complexity. Essentially, there is a huge demand for human-based models that respect PCSK9 physiology. Thus, there is a need to understand others mechanisms of PCSK9, notably for human based models.

Cancer is an abnormal cell growth with the potential to invade or spread to other parts of the body. Cancer treatments, which target several pathways within cancer cells have been developed recently. However, some type of cancers exhibit different known or unknown pathway disruptions or develop treatment resistance. Thus, these cancer treatments are limited since they do not cause cell death in these types of cancer cells and are ineffective at treating various type of cancer. Accordingly, there is a need to develop new approaches and drugs that will be suitable for effective and efficient treatment of cancer and targeting various type of cancer.

### SUMMARY OF THE INVENTION

The invention relates to a method for modulating stem cell proliferation or differentiation comprising a step of contacting said stem cells with an effective amount of an activator or inhibitor of a proprotein convertase subtilisin kexin 9 (PCSK9). In particular, the present invention is defined by the claims.

### DETAILED DESCRIPTION OF THE INVENTION

The inventors have highlighted a new mechanism of PCSK9 in the human induced pluripotent stem cell (hiPSC). More particularly, they have shown that PCSK9 was found highly expressed in hiPSCs and definitive endoderm, involved in the differentiation and its expression was tightly regulated all the way towards hepatocyte fate. Global transcriptomic analyses in hiPSCs showed that PCSK9 inhibition by shRNA and the intracellular PCSK9-R104CN114A mutation negatively regulate the NODAL signaling pathway and its targets. This regulation was manifested in drastic reduction P-SMAD2/total SMAD2 protein level. This was accompanied by reduced proliferation rate where hiPSC-shPCSK9 and hiPSC-R104CN114A demanded >1.3-fold more time to double compared to their control counterparts. They showed that PCSK9 was regulating this signaling pathway through direct physical interaction with DACT2, an intracellular attenuator of NODAL receptor and favoring its protein degradation. Thus, these findings allow to understand the differentiation and proliferation of cells. More particularly, these findings give a new therapeutical approach to treat cancer.

### Method for Modulating Stem Cells Proliferation or Differentiation

Accordingly, in a first aspect, the invention relates to a method for modulating stem cells proliferation or differentiation comprising a step of contacting said stem cells with an effective amount of an activator or inhibitor of a proprotein convertase subtilisin kexin 9 (PCSK9).

As used herein, the term “modulating” refers to stimulating or inhibiting stem cell proliferation, differentiation, or both proliferation and differentiation.

As used herein, the term “stem cells” refers to undifferentiated, or immature cells that are capable of giving rise to multiple, specialized cell types and ultimately to terminally differentiated cells. Unlike any other cells, they are able to renew themselves such that essentially an endless supply of mature cell types can be generated when needed. Due to this capacity for self-renewal, stem cells are therapeutically useful for the regeneration and repair of tissues and organs. Stem cells have the potential for providing benefit in a variety of clinical settings. Stem cells are classified according to their differentiation potential as pluripotent and multipotent. Embryonic stem cells (ESC) are well-known as a pluripotent stem cell, which can be differentiated into almost any type of cells in the organism. Multipotent stem cells such as bone marrow stem cells exist in the adult body. Although their growth and differentiation potential is limited, they are considered to play important roles in self-repair of damaged tissues. In the context of the invention, the term “stem cells” includes human origin or stem cells of non-human mammalian origin. The stem cells may be pluripotent stem cells having the capacity to develop into any cell type, or they may be multipotent stem cells having the capacity to differentiate into several different, final differentiated cell types and derived from a particular tissue or organ, for example, from blood, nerve, skeletal muscle, cardiac muscle, bone marrow, skin, gut, bone, kidney, liver, pancreas, thymus, and the like. Pluripotent stem cells are usually embryonic stem cells in origin and multipotent stem cells include somatic stem cells such as mesenchymal stem cells, bone marrow stem cells, adipose derived stem cells, hemopoietic stem cells, epidermal stem cells and neuronal stem cells.

In a particular embodiment the stem cells are human pluripotent stem cells. As used herein, the term “human pluripotent stem cells” refers to cells that are self-replicating and are derived from human embryos or human fetal tissue. They are known to develop into cells and tissues of the three primary germ layers (ectoderm, mesoderm, and endoderm).

In a particular embodiment, the stem cells are human induced pluripotent stem cells (hiPSCs). The term “human induced pluripotent stem cells” refers to a type of pluripotent stem cell that can be generated directly from human adult cells. Such stem cells can be obtained by performing the method as described by Yamanaka. Yamanaka's lab reported that mouse embryonic fibroblast cells could be reprogrammed in induced pluripotent stem cells also called iPS cells, through retro-viral introduction of four transcriptional factors, Oct4, Sox2, Klf and c-Myc (Takahashi and Yamanaka 2006). In November 2007, Yamanaka's lab and Thomson's lab both reported the generation of human iPS cells from adult human fibroblasts by the combination “Oct4, Sox2, Klf and c-Myc” or “Oct4, Sox2, Nanog, Lin-28”, respectively (Takahashi et al. 2007; Yu et al. 2007). IPS cells were very similar to embryonic stem cells in gene profiling, differentiation potential and epigenetic modifications. They were able to self-renew and differentiate into all mature cell types, including neurons, hematopoietic cells, muscle cells and islet cells.

As used herein, the term “proliferation” refers to an increase in the number of cells of the same type by cell division.

As used herein, the term “differentiation” refers to a developmental process whereby cells become specialized for a particular function, for example, where cells acquire one or more morphological characteristics and/or functions different from that of the initial cell type.

As used herein, the term “PCSK9” refers to proprotein convertase subtilisin/kexin type 9, is a protein that in humans is encoded by the PCSK9 gene and involved in cholesterol homeostasis. The naturally occurring human PCSK9 gene has a nucleotide sequence as shown in Genbank Accession number NM_174936.3 and the naturally occurring human PCSK9 protein has an aminoacid sequence as shown in Genbank Accession number NP_777596.2. The murine nucleotide and amino acid sequences have also been described (Genbank Accession numbers NM_153565.2 and NP_705793.1).

As used herein, the term “activator or inhibitor of a proprotein convertase subtilisin kexin 9” refers to a compound that is capable of stimulating or inhibiting the activity or expression of PCSK9. As used herein the terms “PCSK9 activity” refers to detectable enzymatic, biochemical or cellular activity attributable to PCSK9.

As used herein, the term “activator of PCSK9” refers to a natural or synthetic compound that directly or indirectly increases the PCKS9 activity. It thus refers to any compound able to directly or indirectly increase the transcription, translation, post-translational modification or activity of PCSK9. It includes intracellular as well as extracellular PCSK9 activators including PCSK9 itself.

As used herein, the term “PCSK9 inhibitor” refers to a natural or synthetic compound that directly or indirectly decreases the PCKS9 activity that has a biological effect to inhibit or significantly reduce the activity or expression PCSK9. It thus refers to any compound able to directly or indirectly decrease the transcription, translation, post-translational modification or activity of PCSK9. It includes intracellular as well as extracellular PCSK9 inhibitors.

The activator or inhibitor of PCSK9 activity is a small organic molecule, an aptamer an antibody or a polypeptide.

As used herein the term “small organic molecule” refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

As used herein the term “aptamers” refers to a class of molecule that represents an alternative to antibodies in term of molecular recognition. Aptamers are oligonucleotide or oligopeptide sequences with the capacity to recognize virtually any class of target molecules with high affinity and specificity.

As used herein the term “antibody” refers to any antibody-like molecule that has an antigen binding region, and this term includes antibody fragments that comprise an antigen binding domain such as Fab′, Fab, F(ab′)2, single domain antibodies (DABs or VHH), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art. Particularly, in the context of the invention, the antibody is a single domain antibody. The term “single domain antibody” has its general meaning in the art and refers to the single heavy chain variable domain of antibodies of the type that can be found in Camelid mammals which are naturally devoid of light chains. Such single domain antibody are also called VHH or “Nanobody®”. For a general description of (single) domain antibodies, reference is also made to the prior art cited above, as well as to EP 0 368 684, Ward et al. (Nature 1989 Oct 12; 341 (6242): 544-6), Holt et al., Trends Biotechnol., 2003, 21(11):484-490; and WO 06/030220, WO 06/003388. In the context of the invention, the amino acid residues of the single domain antibody are numbered according to the general numbering for VH domains given by the International ImMunoGeneTics information system aminoacid numbering (http://imgt.cines.fr/). Particularly, in the context of the invention, the antibody is a single chain variable fragment. The term “single chain variable fragment” or “scFv fragment” refers to a single folded polypeptide comprising the VH and VL domains of an antibody linked through a linker molecule. In such a scFv fragment, the VH and VL domains can be either in the VH-linker-VL or VL-linker-VH order. In addition to facilitate its production, a scFv fragment may contain a tag molecule linked to the scFv via a spacer. A scFv fragment thus comprises the VH and VL domains implicated into antigen recognizing but not the immunogenic constant domains of corresponding antibody. In a particular embodiment, the inhibitor of PCS9 activity is an intrabody having specificity for PCSK9. As used herein, the term “intrabody” generally refers to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In a particular embodiment, the inhibitor of PCSK9 activity is Evolocumab commercialized as Repatha® (or AMG 145) by Amgen and has the following formula in the art: C₆₂₄₂H₉₆₄₈N₁₆₆₈O₁₉₉₆S₅₆. In a particular embodiment, the antibody is Alirocumab commercialized as Praluent (REGN727 or SAR2365553) by Sanofi and Régéneron Pharmaceuticals and has the following formula in the art: C₆₄₇₂H₉₉₉₆N₁₇₃₆O₂₀₃₂S₄₂. In a particular embodiment, the antibody is Bococizumab also called as PF-04950615 or RN316 developed by PFIZER (phase 3 ongoing). In a particular embodiment, the antibody is LGT-209 developed by Novartis (phase 2 ongoing). In a particular embodiment, the antibody is RG-7652 developed by Roche (phase 2 ongoing). In a particular embodiment, the antibody is LY3015014 developed by Eli Lilly (phase 2 ongoing).

As used herein, the term “polypeptide” refers to a polypeptide that specifically bind to PCSK9, can be used as a PCSK9 activator or inhibitor that bind to and activate or sequester the PCSK9 protein, thereby stimulating or preventing it from signaling. Polypeptide refers both short peptides with a length of at least two amino acid residues and at most 10 amino acid residues, oligopeptides (11-100 amino acid residues), and longer peptides (the usual interpretation of “polypeptide”, i.e. more than 100 amino acid residues in length) as well as proteins (the functional entity comprising at least one peptide, oligopeptide, or polypeptide which may be chemically modified by being glycosylated, by being lipidated, or by comprising prosthetic groups). The definition of polypeptides also comprises native forms of peptides/proteins in mycobacteria as well as recombinant proteins or peptides in any type of expression vectors transforming any kind of host, and also chemically synthesized peptides. In a particular, the PCSK9 activity inhibitor is an intracellular peptide. Typically, intracellular peptide disturbs transmission of signals of PCSK9 mainly in the cytosol, mitochondria, and/or nucleus. In a particular embodiment, the polypeptide against PCSK9 activity is BMS-962476 as characterized by the amino acid sequences disclosed in WO 2011130354. This polypeptide is also described in Mitchell et al 2010 (J Pharmacol Exp Ther. 2014 August; 350(2):412-24. doi: 10.1124/jpet.114.214221. Epub 2014 Jun. 10.)

In a particular embodiment, the PCSK9 inhibitor is an inhibitor of PCSK9 expression. An “inhibitor of PCSK9 expression” refers to a natural or synthetic compound that has a biological effect to inhibit or significantly reduce the expression of the gene encoding for PCSK9. Typically, the inhibitor of PCSK9 expression has a biological effect on one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

In some embodiments, the inhibitor of PCSK9 expression is an antisense oligonucleotide. Anti-sense oligonucleotides, including anti-sense RNA molecules and anti-sense DNA molecules, would act to directly block the translation of PCSK9 mRNA by binding thereto and thus preventing protein translation or increasing mRNA degradation, thus decreasing the level of PCSK9 proteins, and thus activity, in a cell. For example, antisense oligonucleotides of at least about 15 bases and complementary to unique regions of the mRNA transcript sequence encoding PCSK9 can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

In a particular embodiment, the inhibitor of PCSK9 expression is a shRNA. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound.

In some embodiments, the inhibitor of PCSK9 expression is a small inhibitory RNAs (siRNAs). PCSK9 expression can be reduced by contacting the subject or cell with a small double stranded RNA (dsRNA), or a vector or construct causing the production of a small double stranded RNA, such that PCSK9 expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). In a particular embodiment, the siRNA is ALN-PCS02 developed by Alnylam (phase 1 ongoing).

In some embodiments, inhibitor of PCSK9 expression is a ribozyme. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of PCSK9 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

In some embodiments, the inhibitor of PCSK9 expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

DACT2 has been described as a tumor suppressor, as the restoration of its expression in tumor cells inhibits their proliferation. However, the function of DACT2 in the proliferation or differentiation of stem cells has never been described. For the first time, the inventors of the present invention have demonstrated that PCSK9 inhibition by shRNA and the intracellular PCSK9-R104CN114A mutation negatively regulate the NODAL signaling pathway and its targets. Particularly, they showed that PCSK9 was regulating this signaling pathway through direct physical interaction with DACT2, an intracellular attenuator of NODAL receptor and favoring its protein degradation.

Accordingly, the invention relates also to a method for modulating stem cells proliferation or differentiation comprising a step of contacting said stem cells with an effective amount of an activator or inhibitor of DACT2 activity.

As used herein, the term “DACT2” refers to dishevelled binding antagonist of beta catenin 2. It is involved in regulation of intracellular signaling pathways during development. Negatively regulates the Nodal signaling pathway, possibly by promoting the lysosomal degradation of Nodal receptors, such as TGFBR1.

The naturally occurring human DACT2 gene has a nucleotide sequence as shown in Genbank Accession number NM_001286350.1 and the naturally occurring human DACT2 protein has an amino acid sequence as shown in Genbank Accession number NP_001273279.1. The murine nucleotide and amino acid sequences have also been described (Genbank Accession numbers NM_172826.3 and NP_766414.3).

As used herein, the term “activator or inhibitor DACT2” refers to a compound that is capable of stimulating or inhibiting the activity or expression of DACT2. As used herein the terms “DACT2 activity” refers to detectable enzymatic, biochemical or cellular activity attributable to DACT2.

The activator or inhibitor of DACT2 activity is a small organic molecule, an aptamer an antibody or a polypeptide as described above.

In a particular embodiment, the method according to the invention is suitable to replace damaged cells and treat a disease. For example, the method according to the invention is suitable for treatment of extensive burns, restore the blood system in patients with leukaemia and other blood disorders.

Stem cells as obtained according to the invention may also be used to replacing cells lost in many other devastating diseases for which there are currently no sustainable cures. For example, the stems cells of the invention may be used to correct parts of organs that do not work properly (e.g kidney); to transplant the cells into people with type 1 diabetes. Other medical conditions that may potentially be treated with stem cells include: traumatic spinal cord injury, stroke, severe burns, rheumatoid arthritis, heart diseases, hearing loss, retinal diseases, Huntington's disease or Parkinson's disease.

### Method for Modulating Cancer Stem Cells Proliferation or Differentiation

In a second aspect, the invention relates to a method for modulating cancer stem cells proliferation or differentiation comprising a step of contacting said cancer stem cells with an effective amount of an inhibitor of a proprotein convertase subtilisin kexin 9 (PCSK9).

As used herein, the term “cancer stem cells” corresponds to a minor population of self-renewing cancer cells that are responsive of tumor persistence and recurrence since they are likely to be resistant to conventional treatments. Those CSCs have recently been evidenced in solid tumors from various origins including breast, colon head and neck carcinomas and represent a new therapeutic target. It has shown that those CSC express a large number of embryonic antigens which share the expression with human Embryonic Stem Cells (hESCs) or human Induced Pluripotent Stem Cells (hiPSCs). The expression of some of those embryonic antigens has also been found in differentiated cancer cells that are associated with tumorigenesis and/or tumor progression. As used herein, the terms “cancers expressing human stems cells” refer to cancer stem cells expressing a large number of embryonic antigens which share the expression with human embryonic stem cells (hESCs) or induced pluripotent stem cells (iPSCs). Typically, the cancer expressing human stems cells is selected from the group consisting of bladder carcinoma, breast carcinoma, cervical carcinoma, cholangiocarcinoma, colorectal carcinoma, gastric sarcoma, glioma, lung carcinoma, lymphoma, acute and chronic lymphoid and myeloid leukemias, melanoma, multiple myeloma, osteosarcoma, ovarian carcinoma, pancreatic carcinoma, prostate carcinoma, stomach carcinoma, a head tumor, a neck tumor, and a solid tumor.

In a particular embodiment, the inhibitors of PSCK 9 as described above may be used to inhibit PCSK9 activity or expression.

Accordingly, the present invention relates to a method of treating a cancer in a subject in need thereof comprising a step of administering to said subject a therapeutically effective amount of a proprotein convertase subtilisin kexin 9 (PCSK9) inhibitor.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the terms “cancer”, “tumor”, “cancerous” or malignant” in a subject refers to the presence of cells possessing characteristics typical of cancer-causing cells, such as uncontrolled proliferation, immortality, metastatic potential, rapid growth and proliferation rate, and certain characteristic morphological features. Examples of cancers that may be treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxo sarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcino sarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

As used herein, the term “subject” refers to any mammals, such as a rodent, a feline, a canine, and a primate. Particularly, in the present invention, the subject is a human afflicted with or susceptible to be afflicted with a cancer. In a particular embodiment, the subject suffers from liver cancer. In a particular embodiment, the subject suffers from colon cancer.

The term “PCSK9 inhibitor” refers to a natural or synthetic compound that has a biological effect to inhibit or significantly reduce the activity or expression PCSK9.

In a particular embodiment, the inhibitors of PSCK 9 as described above may be used to inhibit PCSK9 activity or expression.

By a “therapeutically effective amount” is meant a sufficient amount of the single domain antibody of the invention or the polypeptide of the invention to treat the disease (e.g. cancer) at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The PCSK9 inhibitors as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In some embodiments, the PCSK9 inhibitor according to the invention is administered to the subject in combination with another classical treatment for treating cancer. Typically, the classical treatment includes chemotherapy, radiotherapy, and immunotherapy.

In some embodiments, the subject is administered with a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; amino levulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and phannaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and phannaceutically acceptable salts, acids or derivatives of any of the above.

In some embodiments, the subject is administered the inhibitors of PCSk9 as described above in combination with another targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names. In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo [3,4-f][1,6]naphthyridin-3 (2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In certain embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

In some embodiments, the subject is administered with an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation).

Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention.

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen express cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

In some embodiments, the immunotherapeutic agent is an immune checkpoint inhibitor. As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD1 with its ligands PDL1 and PDL2 (Pardoll, Nature Reviews Cancer 12: 252-264, 2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies. In some embodiments, the immune checkpoint inhibitor is an antibody selected from the group consisting of anti-CTLA4 antibodies (e.g. Ipilimumab), anti-PD1 antibodies (e.g. Nivolumab, Pembrolizumab), anti-PDL1 antibodies, anti-TIM3 antibodies, anti-LAG3 antibodies, anti-B7H3 antibodies, anti-B7H4 antibodies, anti-BTLA antibodies, and anti-B7H6 antibodies. Examples of anti-CTLA-4 antibodies are described in U.S. Pat. Nos. 5,811,097; 5,811,097; 5,855,887; 6,051,227; 6,207,157; 6,682,736; 6,984,720; and 7,605,238. One anti-CTLA-4 antibody is tremelimumab, (ticilimumab, CP-675,206). In some embodiments, the anti-CTLA-4 antibody is ipilimumab (also known as 10D1, MDX-D010) a fully human monoclonal IgG antibody that binds to CTLA-4. Another immune checkpoint protein is programmed cell death 1 (PD-1). Examples of PD-1 and PD-L1 blockers are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In some embodiments, the PD-1 blockers include anti-PD-L1 antibodies. In certain other embodiments, the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224 is a fusion protein of B7-DC; an antibody Fc portion; BMS-936559 (MDX-1105-01) for PD-L1 (B7-H1) blockade. Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). In some embodiments, the immunotherapeutic treatment consists of an adoptive immunotherapy, as described by Nicholas P. Restifo, Mark E. Dudley and Steven A. Rosenberg (“Adoptive immunotherapy for cancer: harnessing the T cell response, Nature Reviews Immunology, Volume 12, April 2012). In adoptive immunotherapy, the patient's circulating lymphocytes, or tumor-infiltrated lymphocytes, are isolated in vitro, activated by lymphokines such as IL-2 and readministered (Rosenberg et al., 1988; 1989). The activated lymphocytes are most preferably be the patient's own cells that were earlier isolated from a blood sample and activated (or “expanded”) in vitro.

In some embodiments, the immunotherapeutic treatment consists of allografting, in particular, allograft with hematopoietic stem cell HSC. The immunotherapeutic treatment may also consist in an adoptive immunotherapy, as described by Nicholas P. Restifo, Mark E. Dudley and Steven A. Rosenberg (“Adoptive immunotherapy for cancer: harnessing the T cell response, Nature Reviews Immunology, Volume 12, April 2012). In adoptive immunotherapy, the subject's circulating lymphocytes, NK cells, are isolated amplified ex vivo and readministered to the subject. The activated lymphocytes or NK cells are most preferably be the subject's own cells that were earlier isolated from a blood or tumor sample and activated (or “expanded”) ex vivo.

In some embodiments, the subject is administered with a radiotherapeutic agent. The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

### Method of Screening

In a further aspect, the invention relates to a method of screening a drug suitable for the modulating stem cells comprising i) providing a test compound and ii) determining the ability of said test compound to activate or inhibit the expression or activity of PCSK9.

Any biological assay well known in the art could be suitable for determining the ability of the test compound to activate or inhibit the activity or expression of PCSK9. In some embodiments, the assay first comprises determining the ability of the test compound to bind to PCSK9. In some embodiments, a population of stem cell is then contacted and activated so as to determine the ability of the test compound to activate or inhibit the activity or expression of PCSk9. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of activating or inhibiting the activity or expression of PCSK9, as determined using in vitro methods described herein, are likely to exhibit similar modulatory capacity in applications in vivo. Typically, the test compound is selected from the group consisting of peptides, petptidomimetics, small organic molecules, antibodies (e.g. intraantibodies), aptamers or nucleic acids. For example the test compound according to the invention may be selected from a library of compounds previously synthesised, or a library of compounds for which the structure is determined in a database, or from a library of compounds that have been synthesised de novo.

The invention will be further illustrated by the following figures and examples. However, these examples and figures should not be interpreted in any way as limiting the scope of the present invention.

### FIGURES

FIG. 1: Cell proliferation assay of hiPSC-shCt and hiPSC-shPCSK9 (upper panel; 3 different passages of each cell line performed independently), hiPSC Control and hiPSC-FL-PCSK9-V5 (overexpression) (middle panel) and UhiPSC-Control and UhiPSC PCSK9-R104CN114A (lower panel, 3 different passages of each cell line performed independently), absorbance values at 48 h, 72 h and 96 h were normalized to the absorbance of cells at 24 h after seeding. (*p<0.05, **p<0.01)

FIG. 2: Cell proliferation assay of Caco2-shCt and Caco2-shPCSK9 (upper panel; 3 different passages of each cell line performed independently), absorbance values at 48 h, 72 h and 96 h were normalized to the absorbance of cells at 24 h after seeding. Doubling time estimated based on the growth rate of Caco2 cell lines (lower panel; n=3 of each). (*p<0.05).

FIG. 3: Cell proliferation assay of HepG2-shCt and HepG2-shPCSK9 (upper panel; 3 different passages of each cell line performed independently), absorbance values at 48 h, 72 h and 96 h were normalized to the absorbance of cells at 24 h after seeding. Doubling time estimated based on the growth rate of HepG2 cell lines (lower panel; n=3 of each). (*p<0.05, **p<0.01).

FIG. 4: The impact of R104CN114A dominant negative PCSK9 mutations on the DACT2 expression. DACT2 gene expression by RT-qPCR (3 different passages). DACT2 and α-tubulin protein levels detected by Western blot (3 different passages) with the corresponding quantification. (*p<0.05).

### EXAMPLE

Material & Methods

1. Cell Culture

The human induced pluripotent stem cells (hiPSC) were reprogrammed from patient-derived urine cells and characterized as previously described (Si-Tayeb et al. 2016). hiPSC were cultured on mouse embryonic fibroblasts (MEFs) in hiPSC medium composed of DMEMF12 (Life Technologies) supplemented with 20% Knockout Serum Replacer (Life Technologies), 0.5% L-Glutamine (Life Technologies) with 0.14% β-mercaptoethanol (Sigma), 1% NEAA and 5 ng/ml fibroblast growth factor 2 (FGF2, Miltenyi) in hypoxia (4% O2, 5% CO2) and manually passed once a week. For feeder free culture conditions, hiPSC colonies were manually picked from MEFs and plated onto plates coated with Matrigel (Corning; 0.05 mg/ml) in StemMACS iPS-Brew medium (Miltenyi). Passages were performed using the Gentle Cell Dissociation Buffer (Stem Cell Technologies).

HepG2 cells and CACO2 cells were culture in DMEM supllumented with non essential amino acids, glutamate and 10% FCS.

3. PCSK9 Silencing

PCSK9 gene expression has been silenced upon lentiviral transduction of specific shRNA (Sigma). The clone TRCN0000075236 cloned into the pLKO.1-Puro vector has been used to target PCSK9 while an unspecific shRNA sequence has been used as control. Upon transduction, hiPS cells K3 (Si-Tayeb, Noto, Sepac, et al. 2010), HEepG2 cells and CACO2 cells were subjected to Puromycin (TOCRIS Bioscience 4089/50) selection using a concentration up to 8 μg/ml.

4. PCSK9 Overexpression

Full length PCSK9 cDNA sequence fused with a V5 tag at the C terminus extremity was integrated in the genomic area of AAVS1 integration sites with specific TALENS and homologous sequence. Upon transfection, hiPSC (Si-Tayeb et al Disease Models and Mechanisms 2016) were subjected to Puromycin (TOCRIS Bioscience 4089/50) selection using a concentration up to 8 μg/ml.

5. Gene Expression Analysis

RNA samples were isolated using the RNeasy Mini Kit (Qiagen). Reverse transcription of 1 μg of RNA into cDNA was conducted using the high-capacity cDNA reverse-transcription kit (Applied Biosystems). Conditions were as follows: 10 min at 25° C., and then 2 hours at 37° C. Quantitative Polymerase Chain Reaction (qPCR) studies were conducted in triplicate using the brilliant III Ultra-Fast Master Mix with high ROX (Agilent). Primers are listed in (Table2). Each qPCR included 2 s at 50° C., 10 s at 95° C. followed by 40 cycles of 15 s at 95° C., and 60 s at 60° C. Cycle threshold was calculated by using default settings for the real time sequence detection software (Applied Biosystems).

6. Protein Expression Analysis

hiPSC were initially lysed in a buffer composed of 150 mM NaCl, 25 mM Tris Base, 1 mM EDTA (ethylene diaminetetraacetic acid), and 1% NP-40 (Nonidet P-40) at pH 7.4 and containing a cocktail of protease inhibitors (Sigma Aldrich) and phosphatase inhibitors (Sigma Aldrich). Total cell lysates were then passed 10 times through a fine gauge needle followed by sonication (5 pulses for 5 sec each). A protein assay was then carried out against a range of standard bovine serum albumin (BSA) using Pierce™ BCA Protein Assay Kit. The lysates were denatured for 10 min at 70° C. in a mixture of NuPAGE® Sample Reducing Agent (10X) that contains dithiothreitol (DTT) (500 mM) and NuPAGE® LDS Sample Buffer (4X) containing 2% LDS (lithium dodecyl sulfate), 10% glycerol, 141 mM Tris Base, 106 mM Tris HCl, 0.51 mM EDTA, 0.51 mM EDTA, 0.175 mM Phenol Red and pH 8.5. 25 micrograms of each sample were loaded onto a 10% polyacrylamide gel and the proteins were separated by electrophoresis in presence of SDS. After migration, the proteins are transferred to nitrocellulose membrane (Bio-Rad) using Trans-Blot Turbo Transfer System (Bio-Rad). Revelation and quantification was done by Image Lab software (Bio-Rad). The membrane was saturated for one hour in TBS-T buffer (10 mM Tris, NaCl 0.5 mM and 0.1% Tween-20) containing 5% skimmed milk lyophilized. The membrane was then incubated with primary antibody overnight at 4° C. in TBS-T milk. Horseradish peroxidase (HRP)-conjugated secondary antibody staining was performed for 1 h at room temperature (RT) in TBS-T milk. Protein bands were detected using ECL detection system (Bio-Rad).

7. Proliferation Assay

MTT (3-(4,5-dimethylthiazol-2-yl)-2,5-diphenyltetrazolium bromide) tetrazolium assay was used to assess the proliferation of hiPSC. Briefly, 1×104 shRNA-expressing cells or 5×103 PCSK9 LOF and control hiPSCs were plated onto 96-well-plates (previously coated with Matrigel 0.05 mg/ml in quintuplicates. The day after the passage, media was then changed, supplemented with puromycin for the shRNA-expressing cells (TOCRIS Bioscience, 8 μg/ml). Cells were then incubated with 20 μl/well of MTT (Sigma Aldrich M5655) solution (5 mg/ml in sterile PBS) for 3 h at 37° C. The resulting purple-colored formazan crystals were then solubilized using MTT solvent containing 4 mM HCl, 0.1% NP-40 in isopropanol. Finally the absorbance was read at 590 nm using Perkin Elmer VICTOR™ X3 Multilabel Plate Reader. The proliferation rate was monitored over 24 h, 48 h, 72 h and 96 h.

8. Statistical Analysis

Data are expressed as mean±s.e.m. Significant differences between mean values were determined with the Mann-Whitney U-test for comparison of two groups or paired Student's t-test if appropriate.

Results

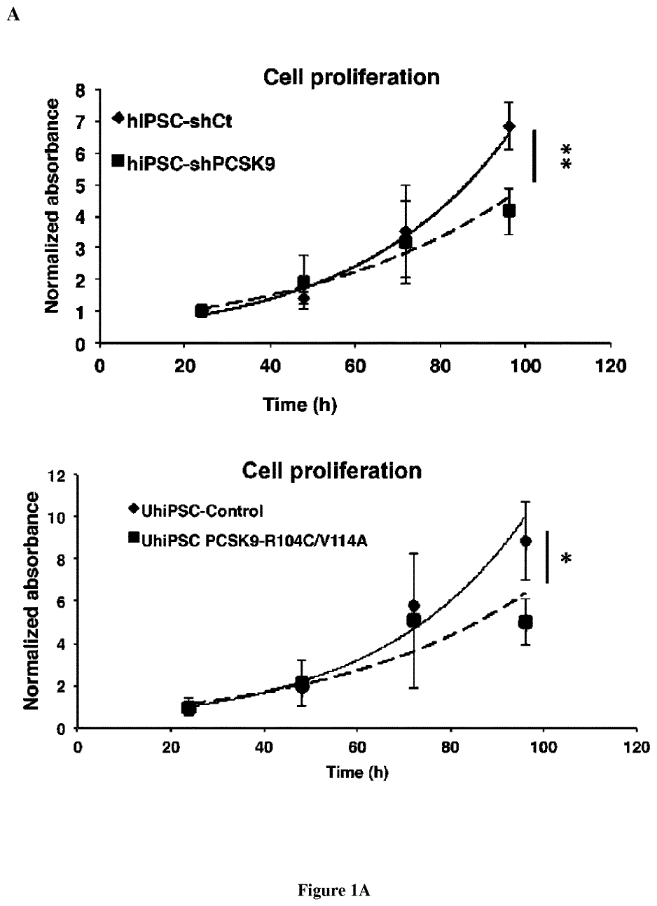

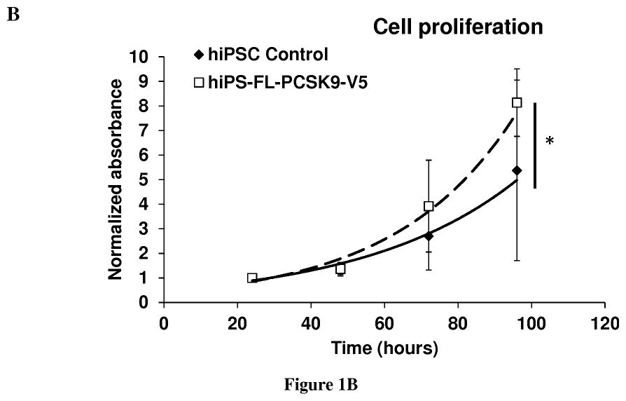

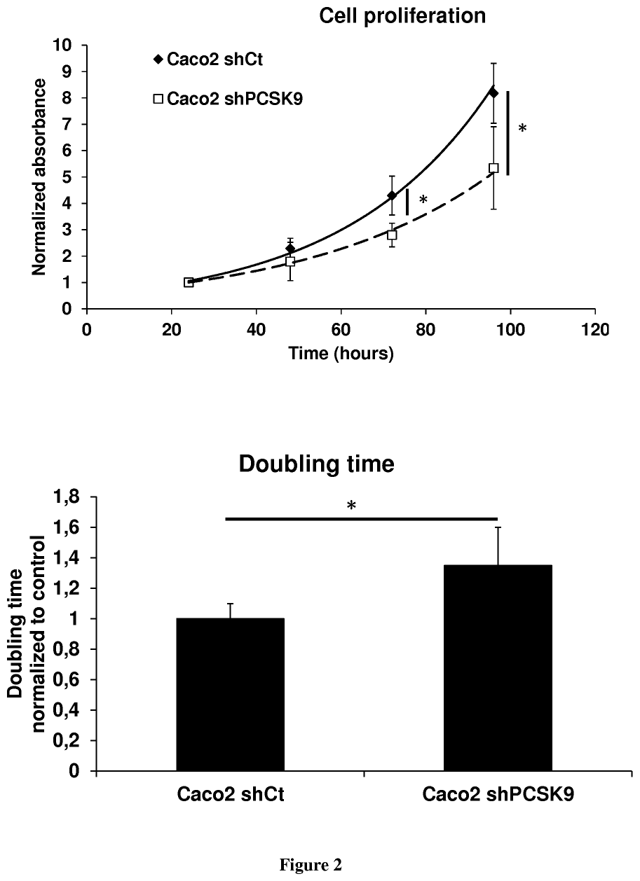

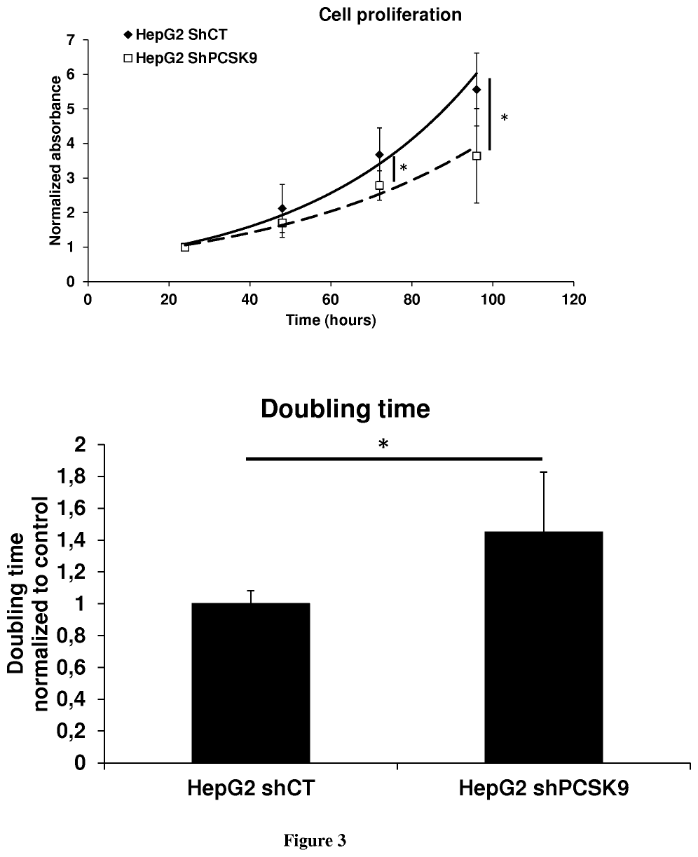

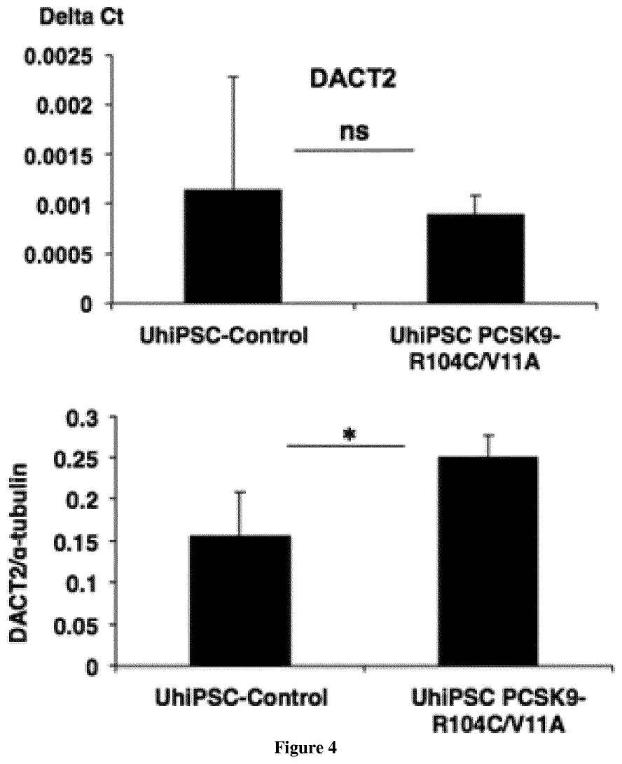

We have showed that PCSK9 inhibition or loss of activity induced a decreased proliferation of hiPSC (FIGS. 1A and 1C) while PCSK9 overexpression increased hiPSC proliferation (FIG. 1B). PCSK9 inhibition in HepG2 cells (FIG. 2) and Caco2 cells (FIG. 3) significantly decreased their proliferation and increased their doubling time rate. Using hiPS cells carrying the dominant negative LOF mutations R104CN114A, we showed that the loss of PCSK9 activity enhanced DACT2 expression and thus decreased SMAD2 signaling (FIG. 4). These data strongly suggest that PCSK9 interacts with the scaffold protein DACT2 (Disheveled antagonist of B-catenin), a tumor supressor, and induces its degradation. Therefore PCSK9 intracellular inhibition led to a decrease in cell proliferation.

### REFERENCES

Throughout this application, various references describe the state of the art to which this invention pertains. The disclosures of these references are hereby incorporated by reference into the present disclosure.

1. A method for modulating stem cells proliferation or differentiation comprising a step of contacting said stem cells with an effective amount of an activator or inhibitor of a proprotein convertase subtilisin kexin 9 (PCSK9).

2. The method according to claim 1 is suitable to replace damaged cells and treat diseases.

3. A method for modulating cancer stem cells proliferation or differentiation comprising a step of contacting said cancer stem cells with an effective amount of an inhibitor of a proprotein convertase subtilisin kexin 9 (PCSK9).

4. A method of treating a cancer in a subject in need thereof comprising a step of administering to said subject a therapeutically effective amount of a proprotein convertase subtilisin kexin 9 (PCSK9) inhibitor.
